(19) United States
(12) Patent Application Publication
(10) Pub. No.: US 2003/0235646 A1
(43) Pub. Date: Dec. 25, 2003

(54) Method for improving quality of meat by supplementing pig diets and concentrates used therefor

(75) Inventors: Jim L. Nelssen, Manhattan, KS (US); Kevin Q. Owen, Canyon, TX (US); Robert D. Goodband, Manhattan, KS (US); Michael D. Tokach, Abilene, KS (US); Steve S. Dritz, Manhattan, KS (US); Brad W. James, Manhattan, KS (US)

Correspondence Address: DARBY & DARBY P.C., Post Office Box 5257, New York, NY, 10150-5257, US

(73) Assignees: Lonza Inc.; Kansas State University Research Foundation

(21) Appl. No.: 10/385,191

(22) Filed: Mar. 10, 2003

Related U.S. Application Data
(60) Provisional application No. 60/365,071, filed on Mar. 15, 2002.

(51) Int. Cl. A61K 38/00; A01N 37/18; A23L 1/00

(52) U.S. Cl. 426/656; 514/2; 426/2

(57) ABSTRACT

The present invention relates to a method of feeding finishing pigs a swine diet which enhances the quality of the meat produced including its drip loss, color, marbling and firmness and thus carcass characteristics. The swine diet includes L-carnitine or salts thereof and ractopamine or salts therof. The amount of the additives present in the diet is such that the quality of the resulting pork and thus carcass characteristics are improved.

### PRIORITY DATA

This application claims priority from U.S. Provisional application Serial No. 60/365,071 filed Mar. 15, 2002, which is hereby incorporated herein by reference.

### BACKGROUND OF THE INVENTION

1. Field of the Invention

The present invention relates to pig farming and relates to the feeding of pigs during both the growing (28 to 61 kg) and finishing (61 to 110 kg) stages. In a particular aspect, it relates to a method of feeding pigs with a feed formulation which enhances the quality of the resulting meat and thus carcass characteristics, as indicated in standard measurements of carcass quality, such as color, marbling and firmness, and also changes in the drip loss, i.e., the amount of liquid or purge that is lost from the cut surface of meat. In addition, the invention relates to concentrates useful for supplementing such feed.

In 1999, ractopamine was approved by the FDA for its use in finishing pigs. Ractopamine belongs to a class of compounds that binds beta-adrenergic receptors and promotes the accretion of muscle protein while reducing body fat. Extensive research has shown that ractopamine improves growth performance and carcass leanness in pigs by directing nutrients away from fat deposition and towards lean deposition. To support the greater lean deposition, pigs need a higher dietary lysine (protein) requirement. The increase in protein deposition for pigs being fed ractopamine is very rapid during the first two weeks after the product is fed. However, because of the increased leanness of the carcasses, as well as changes in intermediary metabolism of pigs fed ractopamine, there is potential for an increased incidence of paler, lighter pork color; softer, less firm cut muscle surface appearance; and increased cut muscle wetness resulting in greater liquid purge loss. These overall effects on pork quality aspects represent negative “side effects” of feeding ractopamine to pigs. Studies have shown consumers avoid and discriminate against pork that is lighter color, softer, and has a watery appearance. These negative effects of ractopamine on pork quality, therefore, represent an economical loss to producers and the meat-packing industry.

2. Description of the Related Art

U.S. Pat. No. 5,124,357 describes using camitine to supplement the diet of a finishing pig.

U.S. Pat. No. 4,690,951 describes using ractopamine to promote growth in animals.

### SUMMARY OF THE INVENTION

The present inventors have surprisingly discovered that supplementing standard finishing pig diets with a combination of L-camitine and ractopamine results in a significant improvement of the resulting meat quality including drip loss, color, marbling and firmness and thus carcass characteristics. This improvement results in pork which is more attractive to the consumer, thus increasing the amount of meat which can be sold by the producer.

Accordingly, one aspect of the present invention is a diet for grower and finishing pigs, where the desired effect of use of this diet is an improvement in the color, marbling and firmness of the meat produced by the pigs.

A further aspect of the present invention is a method of improving the quality of pork including drip loss, color, marbling and firmness that involves feeding finishing pigs a standard diet supplemented with a combination of an effective amount of L-camitine and ractopamine. To achieve the desired effect, L-camitine is added to conventional diets in amounts from about 5 ppm to about 1,000 ppm, and preferably from about 25 ppm to about 100 ppm. Ractopamine is added in amounts from about 2 ppm to about 20 ppm, and preferably from about 5 ppm to about 20 ppm.

Another aspect of the present invention is to ensure that producers of growing and finishing pigs use a diet which has the appropriate ratio of the ractopamine and L-camitine. To achieve the foregoing object, it is preferable to form a concentrate consisting essentially of L-camitine and ractopamine.

The concentrate, when it is blended with the feed, forms a diet containing from about 5 ppm to about 1,000 ppm, preferably from about 25 ppm to about 100 ppm, of L-camitine and from about 2 ppm to about 20 ppm, preferably from about 5 ppm to about 20 ppm, of ractopamine.

The addition of L-carnitine to the diet increases the amount of energy available for protein deposition and increases the response to ractopamine. The practice of the invention ensures the positive effects of ractopamine dosage and dietary L-camitine on growth performance and carcass parameters of growing-finishing pigs, as shown by differences in longissimus quality indicators, such as color, marbling and firmness.

Ractopamine affects pig muscle by directing nutrients away from fat deposition and towards lean deposition. However, in this process, it is believed that changes in intermediary metabolism result in greater lactic acid synthesis, resulting in a lower muscle pH. The low muscle pH changes the chemical bonds within and between muscle cells that result in lighter color, and a soft and watery appearance. Carnitine has been shown to increase pyruvate carboxylase and decrease lactate dehydrogenase in pigs. It appears that the increase in pyruvate carboxylase directs pyruvate away from lactate, thus reducing substrate for lactic acid synthesis postmortem. Furthermore, a decrease in lactate dehyrogenase may delay the onset of postmortem glycolysis. In theory, this would result in an increase in pH, and therefore better water holding capacity and decreased drip loss. Subsequently, meat color would be darker.

### DETAILED DESCRIPTION OF THE INVENTION

All patent applications, patents, and literature references cited in this specification are hereby incorporated by reference in their entirety.

The present invention relates to a diet for finishing pigs which comprises L-camitine and ractopamine.

The use of a concentrate ensures that the L-carnitine and ractopamine are present in concentrations in the diet of the growing and finishing pigs so that the quality of the meat including color, marbling, firmness, and drip loss of the pork and taste, texture and/or moisture of the meat are realized.

Suitable swine to be fed the diet of the present invention include, but are not limited to, all standard breeds of meat-producing pigs such as large white breeds and pigs derived from specific breeding companies, e.g., PIC, Newsham and DeKalb. Further, the addition of L-camitine and ractopamine is advantageous during the finishing stage of development from 15 kg to market weight.

The supplemental camitine is preferably the form L-carnitine. Also suitable for formulation of the diet of the present invention are salts and derivations of carnitine, such as its tartrate, fumarate, magnesium citrate salts, and acetyl L-carnitine. These materials may be encapsulated or protected.

The supplemental ractopamine is preferably the form ractopamine HCl. Also suitable for formulation of the diet of the present invention are salts and derivations of ractopamine. These materials may be encapsulated or protected.

The L-camitine and the ractopamine must be present in an amount effective to achieve the desired effect in the color, marbling, firmness, and the drip loss of the produced pork. Even small differences in the ratio of the two active ingredients have significant effects on attractiveness of the meat to the consumer and on the amount of meat which can be sold by the producer.

Generally, effective concentrations in the pig diet of the L-camitine will range from about 5 ppm to about 1,000 ppm of the feed, preferably from about 25 ppm to about 100 ppm, and most preferably about 50 ppm. Effective concentrations of ractopamine will range from about 5 ppm to about 40 ppm, preferably from about 5 ppm to about 20 ppm, and most preferably about 10 ppm.

The L-camitine and ractopamine are prepared as a concentrate containing from about 40 to 60% of the L-camitine and from about 2 to 60% of ractopamine. The ratio of the L-camitine to ractopamine may be from 20:1 to 1:1, respectively. The concentrate may also contain flowing agents and the like to facilitate the admixing of the L-camitine and ractopamine into the premixes or feed so as to readily form a homogenous blend of the materials.

The concentrate may be blended directly with the feedstock in amounts sufficient to achieve the concentration ranges in the feed set forth above. Alternatively, a premix may be prepared having a ratio of from 20:1 to 1:1 of L-carnitine to ractopamine in amounts sufficient, when mixed with the feed, to result in finished feed compositions containing the concentrations as stated above.

Generally premixes, which include vitamins and minerals, are formed in 100 lb. lots for admixing with one ton of feed. Also, vitamin-free premixes may be formed. Ten lb. units of these are mixed with one ton of feed. Regardless of which method is employed, the premix must contain sufficient L-carnitine and ractopamine to supply sufficient quantities of carnitine and ractopamine in the final feed. The premix may contain from about 500 ppm to about 40%, preferably from about 0.5% to about 40% of L-carnitine or salt thereof, and from about 100 ppm to about 8%, preferably from about 0.1 % to about 8% of ractopamine or salt thereof, based on the total weight of the formulation.

The base diet of the present invention can be any typical swine diet formulated for finishing pigs, particularly finishing pig diets containing ractopamine. For example, a typical diet may include a selection of the ingredients described below. Extensive guidance in formulating diets for the feeding of swine can be found in “Nutrient Requirements of Swine”, Nutrient Requirements of Domestic Animals, Number 3, 9th rev. ed. (National Academy of Science, Washington, D.C. (1988)). Because ractopamine was only approved for use in swine diets in 1999, after the NRC publication, limited information exists regarding nutrient requirements of pigs fed ractopamine. However, it would appear that pigs fed ractopamine require a diet containing greater amounts of lysine (protein) that a typical finishing pig. The increase in lysine content is suggested to be approximately 0.90 to 1.0% total lysine compared with a typical finishing diet containing 0.60 to 0.80% total lysine. (See Table A below.)

In the United States, most pigs are fed a diet consisting of approximately 97% corn and soybean with the remaining 3% consisting of carriers combined with one or more inorganic elements, vitamins, or antimicrobial compounds. For example, a standard diet may contain 79.5% corn; 17.4% soybean meal; 0.9% defluorinated phosphate; 0.65% limestone meal (35% Ca); 0.25% sodium chloride; 0.25% vitamin premix; 0.25% trace element premix; and 0.25% antimicrobial premix. Oats, sorghum and synthetic amino acids are sometimes added. In Europe, corn and soybean meal are generally not as available nor as cost-effective as beans, peas, barley, wheat, rapeseed meal, cassava (tapioca), molasses, fish, bone, and meat meal.

In the United States, most swine producers wean piglets at approximately 21 to 28 days of age, at weights for crossbred piglets of from about 10 pounds (4.8 to 5.2 kg) to 15 pounds (6.5 to 7 kg). Pigs from about one up to about two to three months of age, weighing between about 40 pounds (18 kg) and 110 pounds (50 kg), which are able to be maintained in non-temperature-controlled environments, are called growing pigs. Finishing pigs are pigs being readied for market and range in weight from approximately greater than 110 pounds (50 kg) to 220-310 pounds (100-140 kg) and from greater than about three months up to approximately five to seven months.

The dietary requirements for finishing pigs are different from the dietary requirements for nursery or growing pigs. Nonetheless, the addition of L-carnitine and ractopamine in the disclosed ranges to the diets of finishing pigs has improved the quality of the produced meat.

In formulating diets for finishing pigs, Tables 5 and 6 of the Nutrient Requirements of Swine can be consulted to determine the amino acids, mineral elements, vitamins, and other dietary requirements as a function of weight. Diet components may be, for example, in the following ranges:

TABLE A

| | | FINISHING PIGS FED |
|---|---|---|
| COMPONENT | FINISHING PIGS | RACTOPAMINE |
| Milo or Corn, lb | 1,583-1,757 | 1,360-1,604 |
| Soybean Meal, lb | 185-363 | 328-578 |
| (46.5%) | | |
| Monocalcium | 20-23 | 20-31 |
| Phosphate, lb | | |
| Limestone, lb | 18-19 | 18-19 |
| Salt, lb | 7 | 7 |
| Lysine HCl, lb | 3 | 1-3 |
| Vitamin Premix, lb | 3 | 3 |
| Trace Mineral | 2 | 2 |
| Premix, lb | | |
| Selenium Premix, lb | 1 | 1 |
| Lysine, % | .60-.85 | 0.80-1.15 |
| Ca, % | .65 | .65 |
| P, % | .55 | .55 |

The feed schedule and feed rates used with the present method can be any standard schedule and rate used in the art. The quality of meat after slaughter is determined by the measurement of several standard characteristics, including color, marbling, firmness, taste, texture and moisture exudate.

Color is qualitatively measured using Minolta color-spectrometry, a process which evaluates the Hunter L* and a* values of the meat color, as well as by the saturation index and the Hue Angle value. Hunter L* value measures the lightness of the meat color and varies from 100 for perfect white to 0 for absolute black. Acceptable values for pork range from 35 to 55, with 40-45 values being most preferred by consumers.

The other Hunter value is related to the hue of the color of the meat, with increasing a* values indicating redness and decreasing a values indicating greenness. High quality pork has Hunter a* values which range from 7.5 to 12, and consumers prefer meat with 9-9.5 scores.

Hue Angle value measures the discoloration of meat. Increasing values indicate increasing discoloration of meat. Decreasing values indicate reduction of the discoloration. Acceptable pork Hue Angle values range from 48 to 54, and the preferred values range from 48-52.

The final spectrographic measurement is the saturation index, which is related to the strength of the color of the meat. Acceptable pork saturation index values range from 12 to 15.5, with values from 13-14.5 being preferred. The color of the meat is of particular importance in some export markets, such as Japan, Korea, Asia and South America. Marbling shows intermixture of fat and lean in a cut meat and is measured visually on the scale from 1 to 5, where score 1 indicates no marbling and 5 indicates abundant marbling. Acceptable marbling score ranges from 1 to 3, with 2.5 being preferred.

Drip loss is a measurement of the ability of the sample to retain water. In this procedure, a standardized amount of lean pork muscle, i.e., a cubic centimeter, is weighed, then suspended in an airtight container from a hook for 24 hours. The difference in weight from beginning to end of the 24 hour period represents the amount of water loss or purge. The greater the amount of purge loss, the poorer the pork quality. Changes in the pH of pork associated with postmortem metabolism affect the isoelectric point of the various amino acids in protein, and thus can affect the protein's ability to bind water. Pork with high drip loss is termed in the industry as “exudative” which is undesirable to both meat producers and consumers.

The firmness of the meat is measured visually on the score from 1 to 5, 1 being soft and watery, 5 being firm and dry. The acceptable score is 2-4, and the preferred score is 2.5-3.5. This characteristic of pork has become important due to recent attempts to genetically improve the leanness of the meat. The desired leanness has been accompanied by a negative impact on the meat firmness. As a result, approximately 30% of the meat now going through packing houses has been categorized as PSE (Pale Soft Exudative meat) and is unacceptable to the consumer, thus causing economic loss to the meat producer according to the National Meat Producers Council. Thus, there is an urgent need in the art for means of improving the quality (firmness, color, marbling) and carcass characteristics of the pork.

To supplement the finishing pig feed, and to ensure a proper concentration of each of the components, it is preferred to prepare concentrates rather than admixing the two active ingredients separately. Examples of such concentrates are as follows:

TABLE B

| Formulation | A | B | C | D |
|---|---|---|---|---|
| L-carnitine | 25% | 25% | 50% | 50% |
| Ractopamine.HCl | 5% | 10% | 5% | 10% |
| Calcium carbonate* | 25 | 25 | 20 | 20 |
| Roughage* | 25 | 20 | 15 | 20 |
| Diatomaceous earth* | 20 | 20 | 10 | 10 |
| Mineral oil* | 2% | 2% | 2% | 2% |

*The amounts of the inert ingredients are approximate.

Using the above concentrates, by mixing 0.2 lb. thereof with a ton of feed, the supplement concentrations shown the examples would be achieved. More specifically, Formulation A would result in a feed having 25 ppm of L-camitine and 5 ppm (or 4.5 g/ton) of ractopamine.HCl; Formulation B would result in a feed having 25 ppm of L-camitine and 10 ppm (or 9 g/ton) of ractopamine.HCl; Formulation C would result in a feed having 50 ppm of L-carnitine and 5 ppm (or 4.5 g/ton) of ractopamine.HCl; and Formulation D would result in a feed having 50 ppm of L-camitine and 10 ppm (or 9g/ton) of ractopamine.HCl.

The following examples are representative of the present invention but is not to be considered limiting to the scope of the invention. In these examples, the L-carnitine and the Paylean are separately added to achieve the desired concentration of these two active ingredients.

### EXAMPLE 1

One hundred twenty-six gilts (initially 73.6 lb, PIC C22×L326) were allotted by weight and ancestry in a randomized complete block design to each of the nine experimental treatments arranged in a 3×3 factorial arrangement. There were two pigs/pen and seven replicates/treatment. Pigs were housed in an environmentally controlled building with 4-ft×4-ft slatted-floor pens. Each pen had a one-hole self-feeder and a nipple waterer to allow ad libitum access to feed and water.

Pigs were fed a corn-soybean meal diet composed of the following:

TABLE 1
Basal Diet Composition (As-Fed Basis)^{a}

| Ingredient, % | 73.6 to 164 lb | 164 lb to slaughter |
|---|---|---|
| Corn | 68.41 | 74.50 |
| Soybean meal (46.5% CP) | 26.63 | 22.80 |
| Soybean oil | 2.00 | — |
| Monocalcium phosphate, 21% P | 1.05 | 0.90 |
| Limestone | 1.00 | 0.90 |
| Salt | 0.35 | 0.35 |
| Vitamin premix^{b} | 0.15 | 0.15 |
| Trace mineral premix^{c} | 0.15 | 0.15 |
| L-Lysine HCL | 0.15 | 0.15 |
| Medication^{d} | 0.05 | — |
| DL-Methionine | 0.01 | — |
| Comstarch^{e} | 0.05 | 0.10 |
| Calculated composition | | |
| CP (N × 6.25), % | 18.20 | 16.90 |
| Lysine, % | 1.10 | 1.00 |
| Methionine, % | 0.31 | 0.28 |
| Threonine, % | 0.69 | 0.64 |
| ME, keal/lb | 1,542 | 1,505 |
| Calcium, % | 0.69 | 0.61 |
| Phosphorus, % | 0.60 | 0.55 |

^{a}Diets were formulated to meet or exceed NRC (1998) requirements.
^{b}Contributed per kilogram of complete diet: vitamin A, 8,181 IU; vitamin D₃, 1,322 IU; vitamin E, 35.27 IU; menadione (menadione dimethylpyrimidinol bisulphite), 3.52 mg; vitamin B₁₂, 0.03 mg; riboflavin, 7.94 mg; pantothenic acid, 26.46 mg; niacin, 44.10 mg; choline, 110.3 mg; biotin, 0.04 mg; folic acid, 0.33 mg; and pyridoxine, 3.03 mg.
^{c}Contributed per kilogram of complete diet: Zn (from zinc oxide), 165.3 mg; Fe (from ferrous sulfate), 165.3 mg; Mn (from manganese oxide), 39.7 mg; Cu (from copper sulfate), 16.5 mg; I (from calcium iodate), 0.3 mg; and Se (from sodium selenite), 0.3 mg.
^{d}Provided 44 mg tylosin/kg feed.

L-carnitine (0, 25, or 50 ppm) was added to the diet from 73.6 lb until slaughter (approximately 240 lb). Paylean (a registered trademark of Eli Lilly Company for ractopamine) treatments (0, 4.5, or 9 g/ton) were fed for the last four weeks of the experiment. The basal diet was formulated to contain 1.10% lysine from 73.6 to 164 lb, and 1.00% lysine from 164 lb until the end of the experiment. These quantities of Paylean are equivalent to 0, 5, and 10 ppm of ractopamine in the feed, respectively.

Weights were obtained on every pig and feed disappearance was recorded every fourteen days during the experiment until the last four weeks, at which time measurements were taken weekly to calculate ADG, ADFI, and feed efficiency (F/G). One pig (closest to 240 lb) per pen was selected and slaughtered at the Kansas State University Meat Laboratory. Standard carcass measurements, visual analyses of longissimus muscle color, marbling, and firmness, color spectrophotometry (L*, a*, and b*), drip loss, ultimate pH, and temperature were obtained from each pig at 24-h postmortem.

Data were analyzed as a randomized complete block. Pen was the experimental unit for growth performance data, carcass characteristics, and meat quality measurements. Analysis of variance was performed using the GLM procedure of SAS. Hot carcass weight was used as a covariate in the statistical analysis of backfat, carcass length, loin eye area, and percent lean.

Results and Discussion

Supplementing finishing pig diets with L-carnitine did not affect (P>0.64) growth performance of pigs between 73.6 and 164 lb, but did unexpectedly and positively improve color, marbling, L*, a*/b* ratio and, hue angle. Also, although not significant, there was a numerical tendency for up to a 50% reduction in drip loss when camitine and ractopamine were fed in combination compared with ractopamine alone. It is generally accepted that a 2 unit change in L* values represents a difference in magnitude discemable to the average consumer.

TABLE 2
Carcass Quality Measures of Finishing Pigs Fed Carnitine and Paylean^{a}

| | Paylean, g/ton | | | | | | | | | | | | | | | | |
|---|---|---|---|---|---|---|---|---|---|---|---|---|---|---|---|---|---|
| | 0 | | | 4.5 | | | 9 | | | | | Probability (P<) | | | | | |
| | Carnitine, ppm | | | | | | | | | | Paylean x | | | Paylean | | Carnitine | |
| Item | 0 | 25 | 50 | 0 | 25 | 50 | 0 | 25 | 50 | SEM | Carnitine | Paylean | Carnitine | Linear | Quad | Linear | Quad |
| Visual | 3.35 | 2.78 | 3.14 | 2.57 | 3.28 | 3.49 | 2.57 | 3.00 | 2.85 | 0.25 | 0.02 | 0.15 | 0.18 | 0.99 | 0.08 | 0.11 | 0.82 |
| color^{b} | | | | | | | | | | | | | | | | | |
| Firmness^{b} | 1.93 | 1.65 | 1.93 | 1.79 | 1.93 | 2.05 | 1.79 | 2.15 | 1.79 | 0.24 | 0.43 | 0.88 | 0.88 | 0.66 | 0.81 | 0.66 | 0.81 |
| Marbling^{b} | 2.00 | 1.71 | 1.85 | 1.35 | 2.07 | 1.82 | 1.64 | 2.00 | 1.71 | 0.21 | 0.08 | 0.76 | 0.22 | 0.46 | 0.91 | 0.42 | 0.13 |
| L*^{c} | 55.37 | 58.01 | 56.80 | 60.78 | 56.39 | 55.06 | 61.53 | 58.46 | 57.88 | 1.25 | 0.01 | 0.01 | 0.02 | 0.41 | 0.01 | 0.01 | 0.68 |
| a*^{c} | 7.61 | 6.17 | 6.45 | 5.78 | 6.22 | 7.00 | 6.30 | 6.71 | 6.51 | 0.53 | 0.08 | 0.49 | 0.81 | 0.23 | 0.99 | 0.94 | 0.52 |
| b*^{c} | 15.25 | 14.61 | 15.10 | 15.69 | 14.09 | 14.19 | 15.90 | 14.98 | 15.04 | 0.53 | 0.67 | 0.25 | 0.01 | 0.42 | 0.14 | 0.03 | 0.05 |
| a*/b*^{c} | 0.50 | 0.42 | 0.43 | 0.37 | 0.44 | 0.50 | 0.39 | 0.45 | 0.43 | 0.03 | 0.01 | 0.52 | 0.33 | 0.42 | 0.49 | 0.27 | 0.90 |
| Hue angle | 63.60 | 67.38 | 67.05 | 69.95 | 66.31 | 63.71 | 68.65 | 65.91 | 66.69 | 1.44 | 0.01 | 0.55 | 0.31 | 0.44 | 0.52 | 0.25 | 0.84 |
| Saturation | 17.06 | 15.89 | 16.44 | 16.76 | 15.42 | 15.84 | 17.12 | 16.43 | 16.41 | 0.64 | 0.97 | 0.32 | 0.04 | 0.30 | 0.26 | 0.09 | 0.07 |
| index^{c} | | | | | | | | | | | | | | | | | |
| Drip loss | 2.68 | 2.80 | 2.07 | 3.13 | 1.48 | 1.49 | 3.68 | 2.29 | 2.94 | 0.66 | 0.47 | 0.16 | 0.06 | 0.33 | 0.09 | 0.04 | 0.22 |
| Temp- | 34.72 | 34.83 | 32.98 | 34.39 | 34.38 | 33.80 | 35.72 | 34.15 | 33.76 | 0.83 | 0.60 | 0.74 | 0.04 | 0.97 | 0.44 | 0.01 | 0.62 |
| erature | | | | | | | | | | | | | | | | | |
| (° C.) PH | | | | | | | | | | | | | | | | | |
| 45 m | 6.22 | 6.55 | 6.46 | 6.41 | 6.44 | 6.34 | 6.33 | 6.23 | 6.39 | 0.10 | 0.10 | 0.38 | 0.39 | 0.99 | 0.17 | 0.24 | 0.49 |
| post- | | | | | | | | | | | | | | | | | |
| mortem | | | | | | | | | | | | | | | | | |
| 24 h | 5.75 | 5.79 | 5.76 | 5.79 | 5.86 | 5.86 | 5.71 | 5.79 | 5.78 | 0.04 | 0.91 | 0.01 | 0.04 | 0.01 | 0.06 | 0.07 | 0.08 |
| post- | | | | | | | | | | | | | | | | | |
| mortem | | | | | | | | | | | | | | | | | |

^{a}Values are means of seven or five replications (pig closest to 240 lb in each pen).
^{b}Scoring system of 1 to 5: 2 = grayish pink, traces to slight, or soft and watery; 3 = reddish pink, small to modest, or slightly firm and moist; and 4 = purplish red, moderate to slightly abundant, or firm and moderately dry for color, firmness, and marbling, respectively.
Means were derived from two sample readings per loin. Measures of dark to light (L*), redness (a*), yellowness (^{b}*), red to orange (hue angle), or vividness or intensity (saturation index).

There was a Paylean×L-camitine interaction (P<0.02) for visual color, L*, a*/b* ratio, and Hue angle as shown in Table 2.

L-carnitine did not improve visual color scores in control pigs, but L-carnitine did improve visual color when 4.5 or 9 g/ton of Paylean was fed. Pigs fed increasing levels of L-camitine had lower L* values when fed with 4.5 or 9 g/ ton of Paylean, resulting in a darker colored longissimus muscle measured at the 10^{th} rib. Pigs fed L-camitine and 4.5 or 9 g/ton Paylean, but not control pigs, had lower a*/b* and Hue angle values.

Measurements of b* decreased (quadratic, P<0.05) with increasing levels of L-camitine. The saturation index measured on the longissimus muscle tended to decrease (quadratic, P<0.07) with increasing levels of L-camitine. Drip loss measured 48 hours postmortem and temperature at 45 minutes postmortem decreased (linear, P<0.04) with increasing levels of L-camitine. Twenty-four hour pH increased and then decreased (quadratic, P<0.06) with increasing levels of Paylean and was highest for pigs fed 4.5 g/ton. Ultimate (24-h) pH also increased (linear, P<0.07) with increasing levels of L-camitine.

It is hypothesized that the improvements in meat quality of pigs fed L-carnitine in combination with Paylean may be the result of L-camitine's effect on the pigs' metabolic parameters either antemortem or postmortem. L-carnitine has been shown to increase pyruvate carboxylase and decrease lactate dehydrogenase in pigs. An increase in pyruvate carboxylase may direct pyruvate away from lactate, thus reducing substrate for lactic acid synthesis postmortem. Furthermore, a decrease in lactate dehydrogenase may delay the onset of postmortem glycolysis. In theory, this would result in an increase in pH, and therefore better water-holding capacity or decreased drip loss. Subsequently, meat color would then be improved. In addition, because lactic acid build-up is associated with “downer” pigs, L-camitine may also help alleviate the potential of downer pigs when feeding ractopamine. Therefore, because of the known influence of L-carnitine on enzymes involved in lactic acid production, L-carnitine may be able to reduce the number of downer pigs in a commercial production facility by decreasing the lactic acid level in the blood.

### EXAMPLE 2

Because of the dramatic and potentially economically important changes in pork quality observed in the data of Example 1, a second trial was conducted to confirm these observations. This experiment was conducted in the same facility as Example 1 and used the same diet in Table 1. The primary difference was that the 4.5 g/ton ractopamine level was not included; i.e., only 0 or 9 grams/ton ractopamine was combined with either 0, 25, or 50 ppm of added L-camitine. Results listed below in Table 3 confirm the positive effects of feeding L-camitine in combination with ractopamine on pork firmness, selected color traits, and drip loss.

TABLE 3
Carcass Characteristics of Finishing Pigs fed Carnitine and Paylean^{a}

| | Paylean, g/ton | | | | | | | | | | | |
|---|---|---|---|---|---|---|---|---|---|---|---|---|
| | 0 | | | 9 | | | | Probability (P<) | | | | |
| | Carnitine, ppm | | | | | | | Paylean x | | | Carnitine | |
| Item | 0 | 25 | 50 | 0 | 25 | 50 | SEM | Carnitine | Paylean | Carnitine | Linear | Quadratic |
| Visual cobra | 3.20 | 3.10 | 2.90 | 2.75 | 2.75 | 2.80 | 0.16 | 0.52 | 0.02 | 0.72 | 0.93 | 0.43 |
| Firmness^{b} | 2.59 | 2.44 | 2.34 | 1.99 | 2.59 | 2.34 | 0.15 | 0.04 | 0.25 | 0.33 | 0.75 | 0.15 |
| Marbling^{b} | 1.65 | 1.75 | 1.55 | 1.85 | 1.75 | 1.60 | 0.18 | 0.85 | 0.57 | 0.53 | 0.33 | 0.57 |
| L*^{c} | 57.18 | 57.23 | 58.00 | 59.72 | 59.63 | 58.44 | 0.83 | 0.37 | 0.01 | 0.95 | 0.78 | 0.89 |
| a*^{c} | 7.54 | 7.58 | 7.93 | 7.94 | 6.73 | 6.61 | 0.38 | 0.07 | 0.07 | 0.29 | 0.24 | 0.30 |
| b*^{c} | 15.81 | 15.86 | 16.27 | 16.97 | 15.75 | 15.29 | 0.47 | 0.08 | 0.95 | 0.37 | 0.22 | 0.51 |
| a*/b*^{c} | 0.48 | 0.48 | 0.49 | 0.46 | 0.43 | 0.43 | 0.02 | 0.40 | 0.01 | 0.44 | 0.46 | 0.30 |
| Hue angle^{c} | 64.49 | 64.61 | 64.07 | 65.14 | 66.99 | 66.70 | 0.78 | 0.39 | 0.01 | 0.45 | 0.47 | 0.30 |
| Saturation index^{c} | 17.52 | 17.59 | 18.11 | 18.74 | 17.13 | 16.67 | 0.56 | 0.06 | 0.64 | 0.321 | 0.20 | 0.42 |
| Drip loss (%) | 2.04 | 3.07 | 2.73 | 4.85 | 2.47 | 2.82 | 0.64 | 0.02 | 0.48 | 0.15 | 0.32 | 0.56 |
| Temperature (° C.) | | | | | | | | | | | | |
| 5 min | 38.59 | 39.79 | 39.17 | 39.60 | 39.74 | 39.68 | 0.56 | 0.60 | 0.24 | 0.40 | 0.51 | 0.25 |
| 15 min | 40.20 | 39.92 | 39.56 | 40.42 | 40.18 | 40.06 | 0.31 | 0.88 | 0.16 | 0.23 | 0.09 | 0.97 |
| 45 min | 37.72 | 39.03 | 38.73 | 39.43 | 38.35 | 38.76 | 0.69 | 0.18 | 0.16 | 0.23 | 0.73 | 0.93 |
| 1.5 h | 32.91 | 32.87 | 33.17 | 35.99 | 33.51 | 32.65 | 0.72 | 0.04 | 0.06 | 0.07 | 0.05 | 0.46 |
| 3 h | 21.38 | 22.12 | 20.89 | 24.38 | 22.37 | 22.76 | 0.63 | 0.10 | 0.01 | 0.26 | 0.11 | 0.85 |
| 6 h | 10.74 | 11.28 | 11.22 | 12.04 | 11.09 | 10.97 | 0.48 | 0.20 | 0.47 | 0.82 | 0.55 | 0.89 |
| pH | | | | | | | | | | | | |
| 0 min | 7.14 | 7.13 | 7.21 | 7.16 | 7.16 | 7.21 | 0.05 | 0.94 | 0.76 | 0.37 | 0.23 | 0.47 |
| 5 min | 6.93 | 6.84 | 6.82 | 6.79 | 6.80 | 6.94 | 0.07 | 0.17 | 0.76 | 0.66 | 0.74 | 0.40 |
| 15 min | 6.55 | 6.60 | 6.58 | 6.59 | 6.47 | 6.49 | 0.07 | 0.48 | 0.35 | 0.84 | 0.62 | 0.76 |
| 45 min | 6.16 | 6.16 | 6.02 | 6.14 | 6.21 | 6.13 | 0.10 | 0.82 | 0.57 | 0.54 | 0.44 | 0.43 |
| 1.5 h | 5.95 | 5.91 | 5.97 | 5.89 | 5.95 | 5.92 | 0.10 | 0.87 | 0.74 | 0.96 | 0.77 | 0.94 |
| 3 h | 5.77 | 5.76 | 5.88 | 5.59 | 5.67 | 5.69 | 0.08 | 0.77 | 0.01 | 0.33 | 0.15 | 0.73 |
| 6 h | 5.76 | 5.70 | 5.70 | 5.61 | 5.66 | 5.68 | 0.04 | 0.23 | 0.06 | 0.98 | 0.90 | 0.89 |
| 24 h | 5.64 | 5.61 | 5.60 | 5.58 | 5.64 | 5.59 | 0.02 | 0.19 | 0.57 | 0.46 | 0.60 | 0.26 |

^{a}Values are means often replications (one pig selected randomly from each pen).
^{b}Scoring system of 1 to 5: 2 = grayish pink, traces to slight, or soft and watery; 3 = reddish pink, small to modest, or slightly firm and moist; and 4 = purplish red, moderate to slightly abundant, or firm and moderately dry for color, firmness, and marbling, respectively.
^{c}Means were derived from two sample readings per loin. Measures of dark to light (L*), redness (a*), yellowness (b*), red to orange (hue angle), or vividness or intensity (saturation index).

The data set forth above show that L-camitine improves meat quality in pigs fed Paylean.

### EXAMPLE 3

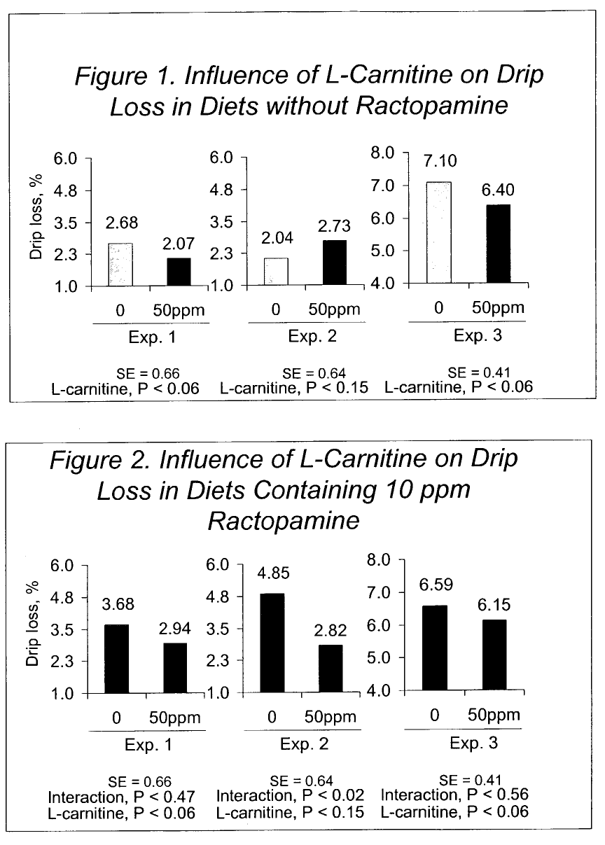

Similar results of the combined effects of L-carnitine and ractopamine on pork quality indicators have been observed in follow-up studies. FIGS. 1 and 2 summarize the effects of added L-carnitine with or without ractopamine. FIG. 1 shows that in three different experiments added camitine at 50 ppm has a small positive effect on drip loss in two of the three studies. However, as shown in FIG. 2, adding camitine to diet containing ractopamine tends to have a greater net positive effect on reducing (improving) pork drip loss.

What is claimed is:

1. A method for improving the quality of the meat produced by a finishing pig comprising: the step of feeding to said finishing pig a diet supplemented with effective amounts of L-carnitine or a salt thereof and ractopamine or a salt thereof.

2. The method of claim 1 wherein the quality of the meat to be improved is drip loss, color, marbling or firmness of the meat.

3. The method of claim 1 wherein the carnitine is L-carnitine.

4. The method of claim 1 wherein the ractopamine is ractopamine.HCl.

5. The method of claim 1 wherein the effective amount of L-carnitine is from about 5 to 1,000 ppm of the feed and the effective amount of ractopamine is from about 2 to 20 ppm of the feed.

6. The method of claim 1 wherein the effective amount of L-carnitine is from about 5 to 100 ppm of the feed and the effective amount of ractopamine is from about 5 to 20 ppm of the feed.

7. A method for improving the quality of the meat produced by a finishing pig which comprises: admixing L-carnitine or a salt thereof and ractopamine or a salt thereof with a flowing agent to form a feed supplement concentrate; blending said feed supplement concentrate with a finishing pig diet to form a supplemented finishing pig diet; said supplemented finishing pig diet containing from about 5 to 1,000 ppm of carnitine and from about 2 to 20 ppm of ractopamine; said L-carnitine and ractopamine being in a weight ratio of from 20:1 to 1:1.

8. The method of claim 7 wherein the quality of the meat to be improved is drip loss, color, marbling or firmness of the meat.

9. The method of claim 7 wherein the camitine is L-carnitine.

10. The method of claim 7 wherein the ractopamine is ractopamine.HCl.

11. The method of claim 7 wherein the effective amount of L-camitine is from about 5 to about 100 ppm of the feed and the effective amount of ractopamine is from about 5 to about 20 ppm of the feed.

12. A premix for use in preparing a finishing pig feed which comprises feed, L-carnitine or a salt thereof and ractopamine or a salt thereof, the latter two components in a weight ratio of from 20:1 to 1:1.

13. The premix of claim 12 wherein the amount of the L-camitine or salt thereof is from about 500 ppm to about 40% and the amount of the ractopamine or salt thereof is from about 100 ppm to about 8%, based on the total weight of the formulation.

14. The premix of claim 12 wherein the amount of the L-camitine or salt thereof is from about 0.5 % to about 40% and the amount of the ractopamine or salt thereof is from about 0.1% to about 8%, based on the total weight of the formulation.

15. The premix of claim 14 wherein the L-camitine and ractopamine.HCl are in the formulation.

16. A feed supplement concentrate comprising L-carnitine, ractopamine, and a flow agent, wherein the L-carnitine and ractopamine are in a weight ratio of from 20:1 to 1:1.

17. The feed supplement concentrate of claim 16 wherein the L-camitine is from 40 to 60% and the ractopamine is from 2 to 60% of the total composition.
